(19) United States
(12) Patent Application Publication
(10) Pub. No.: US 2026/0337597 A1
(43) Pub. Date: Oct. 29, 2026

(54) DIRECT AFFINITY TO SLALOM CHROMATOGRAPHY METHODS AND SYSTEMS THEREOF

(71) Applicant: Waters Technologies Corporation, Milford, MA (US)

(72) Inventors: Fabrice Gritti, Franklin, MA (US); Kevin Wyndham, Worcester, MA (US); Beatrice Muriithi, Attleboro, MA (US); Martin Gilar, Franklin, MA (US); Nathan Canniff, Concord, MA (US)

(73) Assignee: Waters Technologies Corporation, Milford, MA (US)

(21) Appl. No.: 19/643,942

(22) Filed: Apr. 10, 2026

Related U.S. Application Data
(60) Provisional application No. 63/794,849, filed on Apr. 25, 2025.

(51) Int. Cl. C12N 15/10; B01D 15/18; B01D 15/38; B01D 15/42; B01J 20/24; B01J 20/288; B01J 20/32; G01N 30/02; G01N 30/16

(52) U.S. Cl. CPC C12N 15/101; B01D 15/1871; B01D 15/3809; B01D 15/3823; B01D 15/424; B01J 20/24; B01J 20/288; B01J 20/3208; B01J 20/3217; B01J 20/3274; B01J 20/3293; G01N 30/16; B01J 2220/44; B01J 2220/54; G01N 2030/027

(57) ABSTRACT

The present disclosure is directed to methods of performing direct affinity-slalom chromatography, wherein there is a direct elution of the sample from the affinity chromatography column to the slalom column. The methods described herein allow for rapid and robust purification of target oligonucleotides from heterogeneous samples, and mitigate the need for complicated valve switching, buffer exchange, or other sample manipulation.

### CROSS-REFERENCE TO RELATED APPLICATIONS

This application claims priority from and the benefit of U.S. Provisional Application No. 63/794,849 filed Apr. 25, 2025. The entire contents of this application are incorporated herein by reference.

### FIELD OF INVENTION

The present disclosure relates generally to methods of performing direct affinity chromatography to slalom chromatography, particularly using a direct fluidic connection between the affinity chromatography column and the slalom chromatography column.

### BACKGROUND

Chromatography methods are used across the pharmaceutical, biotechnology, and chemical industries for the separation and characterization of target oligonucleotides in heterogeneous samples. Multiple chromatography methods may be necessary in a particular workflow. For example, the use of affinity chromatography to isolate a target oligonucleotide based on its affinity to an affinity agent, and slalom chromatography to isolate a target oligonucleotide based on its response to high-flow conditions. Typically, the use of both affinity chromatography and slalom chromatography requires separate or complex chromatography systems due to technical incompatibilities, including buffer and pressure incompatibilities. In particular, the different solvent conditions required for affinity chromatography and slalom chromatography greatly increase the processing required for a sample purification method requiring both affinity and slalom chromatography.

### SUMMARY OF INVENTION

Disclosed herein are methods of performing tandem affinity chromatography and slalom chromatography using a direct elution method from the affinity chromatography column to the slalom column (direct affinity-slalom chromatography; or alternatively direct affinity-slalom). The present methods are particularly suitable for separating a first group oligonucleotides (e.g., a group of ssRNA, a group of dsRNA, a group of ssDNA, or a group of dsDNA) from a second group of oligonucleotides (e.g., a group of ssRNA, a group of dsRNA, a group of ssDNA, or a group of dsDNA), and then further separating each group based on the principles of slalom chromatography.

Accordingly, in a first aspect, the present disclosure provides for a method of purifying a sample. The method comprises:
a) loading the sample onto an affinity chromatography column in direct fluidic connection to a slalom chromatography column, wherein the sample comprises a first group of oligonucleotides and a second group of oligonucleotides;
   wherein the affinity chromatography column comprises:
a plurality of affinity chromatography particles, wherein the plurality of affinity chromatography particle includes a plurality of nonporous polymer particles, wherein each particle within the plurality of nonporous polymer particles include a polymer core and a hydrophilic surface on an outer layer of the polymer core; and one or more affinity agents conjugated to a surface of each particle of the plurality of affinity chromatography particles, wherein the first group of oligonucleotides comprises a stronger affinity for the one or more affinity agents than the second group of oligonucleotides;
   and the slalom chromatography column comprises a plurality of slalom chromatography particles;
b) eluting the second group of oligonucleotides from the affinity chromatography column directly to the slalom chromatography column and retaining the first group of oligonucleotides in the affinity chromatography column in the presence of a mobile phase;
c) providing an elution injection to the affinity chromatography column, thereby releasing the first group of oligonucleotides from the affinity chromatography column;
d) eluting the first group of oligonucleotides from the affinity chromatography column directly to the slalom chromatography column in the presence of the mobile phase; and
e) detecting an injection breakthrough and an eluent from the slalom chromatography column at a detector;
   wherein the injection breakthrough forms at least one distinct peak and the eluent form at least one distinct peak.

In some embodiments, the nonporous polymer particles have an average particle size between 1.0 μm to 10 μm. In certain embodiments the slalom chromatography columns also have an average particle size between 1.0 to 10 μm. In some embodiments, the one or more affinity agents are conjugated directly to a surface of each particle of the plurality of affinity chromatography particles. In some embodiments, the one or more affinity agents are conjugated to the surface of each particle of the plurality of affinity chromatography particles covalently. In some embodiments, the one or more affinity agents are conjugated indirectly to the surface of each particle of the plurality of affinity chromatography particles through one or more conjugating molecules. In some embodiments, the one or more conjugating molecules comprise streptavidin. In some embodiments, the one or more affinity agents are biotinylated.

In some embodiments, the one or more affinity agents comprise an oligonucleotide or an oligonucleotide specific antibody. In some embodiments, the oligonucleotide is a poly-T oligonucleotide. In some embodiments, the oligonucleotide specific antibody is a J2 monobody. In some embodiments, the plurality of affinity chromatography particles are from about 1 μm to about 10 μm.

In some embodiments, the first group of oligonucleotides comprises a single stranded RNA (ssRNA), and the second group of oligonucleotides comprises a double stranded RNA (dsRNA). In some embodiments, the first group of oligonucleotides comprises a plurality of oligonucleotide lengths, wherein each of the plurality of oligonucleotide lengths are at least 10 bases. In some embodiments, the first group of oligonucleotides comprises a plurality of oligonucleotide lengths, wherein each of the plurality of oligonucleotide lengths is from about 40 bases to about 40000 bases. In some embodiments, the second group of oligonucleotides comprises a plurality of oligonucleotide lengths, wherein each of the plurality of oligonucleotide lengths are at least 10 bases. In some embodiments, the second group of oligonucleotides comprises a plurality of oligonucleotide lengths, wherein each of the plurality of oligonucleotide lengths is from about 40 bases to about 40000 bases.

In some embodiments, the sample injection comprises a sample injection volume of from about 0.1 μL to about 100 μL. In some embodiments, the sample injection volume is about 1 μL. In some embodiments, step b) comprises a first mobile phase flow rate of from about 0.1 mL/min to about 1 mL/min. In some embodiments, the first mobile phase flow rate is about 0.1 mL/min.

In some embodiments, the method further comprises a wait time of from about 0.1 min to 20 min between steps b) and c). In some embodiments, the wait time is about 2.5 min. It will be understood, that in some embodiments the wait time between a sample injection and an elution injection may be varied depending on the characteristics of a specific sample. In some embodiments, the mobile phase is supplied at a flow rate of from about 0.1 mL/min to about 1 mL/min for the duration of the wait time. In some embodiments, the mobile phase is supplied at a flow rate of about 0.8 mL/min for the duration of the wait time.

In some embodiments, the injection breakthrough is detected prior to step c). In some embodiments, the injection breakthrough comprises the first group of oligonucleotides comprises detecting at least one distinct peak. In some embodiments, the detector is the same detector used in step e).

In some embodiments, the elution injection comprises an elution injection volume of from about 0.1 μL to about 100 μL. In some embodiments, the elution injection volume is about 50 μL. In some embodiments, the elution injection comprises a first elution injection flow rate of from about 0.1 mL/min to about 1 mL/min. In some embodiments, the first elution injection flow rate is about 0.1 mL/min.

In some embodiments, the method further comprises modulating the mobile phase flow rate to a second elution injection flow rate after step d), wherein the second elution injection flow rate is from about 0.1 mL/min to about 1 mL/min. In some embodiments, the first elution injection flow rate is about 0.8 mL/min. In some embodiments, the eluent comprises the second group of oligonucleotides.

In some embodiments, the affinity chromatography column and the slalom chromatography column are connected to a high-performance liquid chromatography (HPLC) system, ultra-high performance liquid chromatography (UHPLC) system, or fast protein liquid chromatography (FPLC) system. In some embodiments, the detector is an ultraviolet spectroscopy detector, a fluorescence spectroscopy detector, a multi-angle light scattering detector, a charged aerosol detector, and/or a mass spectrometry detector.

In some embodiments, the direct fluidic connection is a zero dead volume union. In some embodiments, the eluent from the slalom chromatography column results in a zone retention factor of less than 0.35.

In a second aspect, disclosed herein is a chromatographic system comprising:
a) an affinity chromatography column and a slalom chromatography column, wherein the affinity chromatography column and the slalom column are connected via a zero dead volume union, wherein the slalom column is positioned downstream of the affinity chromatography column; wherein the affinity chromatography column comprises:
   (i) a plurality of affinity chromatography particles, wherein the plurality of affinity chromatography particle includes a plurality of nonporous polymer particles, wherein each particle within the plurality of nonporous polymer particles include a polymer core and a hydrophilic surface on an outer layer of the polymer core; and
   (ii) one or more affinity agents conjugated to a surface of each particle of the plurality of affinity chromatography particles,
   (iii) and the slalom chromatography column comprises
   (iv) a plurality of slalom chromatography particles;
b) column injector positioned upstream of the affinity chromatography column; and
c) tubing in fluidic connection with the slalom column positioned downstream of the slalom column.

In some embodiments, the nonporous polymer particles have an average particle size between 1.0 μm to 10 μm. In certain embodiments the slalom chromatography columns also have an average particle size between 1.0 to 10 μm. In some embodiments, the one or more affinity agents are conjugated directly to a surface of each affinity chromatography particle. In some embodiments, the one or more affinity agents are conjugated to the surface of each affinity chromatography particle covalently. In some embodiments, the one or more affinity agents are conjugated indirectly to the surface of each affinity chromatography particle through one or more conjugating molecules. In some embodiments, the one or more conjugating molecules comprise streptavidin. In some embodiments, the one or more affinity agents are biotinylated.

In some embodiments, the one or more affinity agents comprise an oligonucleotide or an oligonucleotide specific antibody. In some embodiments, the oligonucleotide is a poly-T oligonucleotide. In some embodiments, the oligonucleotide specific antibody is a J2 monobody.

In some embodiments, the chromatographic system further comprises a detector in fluid communication with the tubing downstream of the slalom chromatography column. In some embodiments, the detector is an ultraviolet spectroscopy detector, a fluorescence spectroscopy detector, a multi-angle light scattering detector, a charged aerosol detector, and/or a mass spectrometry detector.

### DEFINITIONS

In order that the methods and technology may be more readily understood, certain terms are first defined. In addition, it should be noted that whenever a value or range of values of a parameter are recited, it is intended that values and ranges intermediate to the recited values are also part of this disclosure. The word “about”, if not defined otherwise, means±5%. It is also to be noted that as used herein and in the claims, the singular forms “a” and “the” include plural references unless the context clearly dictates otherwise.

As used herein, the term “direct fluidic connection” refers to a flow path between one or more column bodies, column injectors, detectors, and/or tubing connecting said elements, that is uninterrupted by a switching valve.

As used herein, the term “zero dead volume union” refers to a union between one or more elements having a direct fluidic connection, wherein the union introduces 1 μL or less of volume to the flow path. A zero dead volume union can be introduced between any two elements having a direct fluidic connection. In preferred embodiments of the present technology, a zero dead volume union is introduced between an affinity chromatography column and a slalom chromatography column having a direct fluidic connection.

As used herein, the term “low volume union” refers to a union between one or more elements having a direct fluidic connection, wherein the union introduces 30 μL or less of volume to the flow path, preferably 20 μL or less, more preferably 10 μL or less. A low volume union can be introduced between any two elements having a direct fluidic connection. In some embodiments of the present technology, a low volume union is introduced between an affinity chromatography column and a slalom column having a direct fluidic connection.

As used herein, the term “direct affinity-slalom chromatography” or “direct affinity-slalom”, used interchangeably, refers to a method of performing chromatography wherein a sample is first separated via an affinity chromatography column and then directly transferred onto a slalom column, and separated by the slalom column. In the direct affinity-slalom methods described herein, the affinity chromatography column is in direct fluidic connection to the slalom chromatography column. That is, any sample injected onto the affinity chromatography column flows through both the affinity chromatography column and the slalom column, as shown in FIG. 1A.

As used herein, the term “eluting the target oligonucleotide from the affinity chromatography column” refers to the use of an elution buffer and elution method (such as, e.g., a gradient elution, a single injection elution, or a step elution) to elute oligonucleotides bound to the affinity column. In some embodiments, the oligonucleotides are eluted in a single narrow peak having a peak volume of between 5-20 μL.

As used herein, the term “conjugate” refers to the linkage of two molecules formed by the chemical bonding of a reactive functional group of one molecule with an appropriately reactive functional group of another molecule. Nonporous polymer particles may have one or more affinity reagents conjugated to the surface of said particles. For example, one or more affinity agents, such as Protein A, may be conjugated directly to a particle via an interaction with an epoxide on the surface of the particle. Alternatively, an affinity agent may be indirectly conjugated to the surface of the nonporous polymer particles via a linker (such as a polyethylene glycol (PEG) linker) or via an interaction with a streptavidin molecule. For the latter instance, one or more streptavidin molecules are conjugated directly to a particle via an interaction with an epoxide on the surface of the particle, which can then bind, via an ionic interaction, to a biotinylated affinity agent.

As used herein, the term “antibody” refers to an immunoglobin molecule that specifically binds to, or is immunologically reactive with, a particular antigen. This includes polyclonal, monoclonal, genetically engineering, and otherwise modified forms of antibodies, including but not limited to chimeric antibodies, camelids, monobodies, humanized antibodies, heteroconjugate antibodies (e.g., bi-, tri-, and quad-specific antibodies, diabodies), and antigen-binding fragments of antibodies, including, for example, Fab′, F(ab′)2, Fab, Fv, and scFv fragments. Unless otherwise indicated, the term “monoclonal antibody” is meant to include both intact molecules as well as antibody fragments that are capable of specifically binding to a target protein. As used herein, the Fab and F(ab′)2 fragments refer to antibody fragments that lack the Fc portion of an intact antibody.

As used herein, the term “polyclonal antibody” refers to an antibody or a population of antibodies that has specificity to one or more antigens (such as, e.g., host cell proteins from a host cell line). A population of polyclonal antibodies recognize one or more distinct epitopes of the one or more antigens.

As used herein, the term “antigen-binding fragment” refers to one or more fragments of an antibody that retain the ability to specifically bind to a target antigen. The antigen-binding function of an antibody can be performed by fragments of a full-length antibody. The antibody fragments can be, for example, a Fab, F(ab′)2, scFv, a camelid, an affibody, a nanobody, an aptamer, or a domain antibody.

As used herein, the term “bispecific antibody” refers to an antibody that is capable of binding at least two different antigens.

The term “nonporous” or “nonporous core” as used herein, refers to a material or a material region (e.g., the core) that has a pore volume that is less than 0.1 cc/g. Preferably, nonporous polymer cores have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

As used herein, the term “slalom chromatography” refers to a chromatographic method performed under high-flow conditions that separates a molecule by size based on hydrodynamic phenomena. The principles of slalom chromatography are further described in Hirabayashi and Kasai, J. Chromatogr. A (1996) 722(1-2):135-42 and Hirabayashi et al., Biochemistry (1990) 29(41):9515-21. The high-flow rates used in slalom chromatography (SC) afford sufficiently high shear rates that result in prolonged extension of the molecules, e.g., nucleic acids, which is important for the separation of the molecules by size. The extension of the molecule can be assessed using the Weissenberg number.

As used herein, the term “nucleic acids” or “nucleic acid molecules” refers to a polymeric molecule comprising two or more nucleotides. Nucleic acids may comprise deoxyribonucleic acids (DNA), ribonucleic acids (RNA), or a combination thereof. DNA comprises the nucleotides cytidine, guanosine, adenosine, and thymidine. RNA comprises the nucleotides cytidine, guanosine, adenosine, and uridine. In some embodiments, the nucleic acid molecules may comprise nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043. Nucleic acid molecules may further be modified at the nucleobase, sugar, or phosphodiester backbone with an array of chemical modifications which are further reviewed in Epple et al., Emerg. Top. Life. Sci. (2021) 5(5):691-697.

Nucleic acid molecules range in length as measured by the number of nucleotides or base pairs. In embodiments of the present technology, the nucleic acids may range from about 5000 base pairs to about 100,000 base pairs. Nucleic acids may be single stranded (e.g., single-stranded DNA (ssDNA) or single-stranded RNA (ssRNA)). In some embodiments, the ssRNA is mRNA. Nucleic acids may be double stranded (e.g., double-stranded DNA (dsDNA) or double-stranded RNA (dsRNA). Double-stranded nucleic acids are made of complementary sequences (e.g., base-paired sequences) as is known in the art and would be readily understood by one of ordinary skill.

Nucleic acids may be present in different topologies, including linear and circular conformations. In some embodiments, circular nucleic acids may be supercoiled, in which the circular nucleic acid molecule undergoes additional twist strain.

As used herein, the term “porous” refers to a material that has a pore volume that is greater than 0.1 cc/g. Preferably, porous polymers have a pore volume that is greater than 0.1 cc/g (e.g., 0.5 cc/g). As used herein, the term “nonporous” refers to a material that has a pore volume that is less than 0.1 cc/g. As used herein, the term “superficially porous” refers to a material having a nonporous region (e.g., a core, a solid-core) and a porous region (e.g., a shell or outer region surrounding the core). Preferably, nonporous materials (or the nonporous region of a material) have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA).

As used herein, the term “distinct peak” refers to a peak in a chromatogram, e.g., a detected analyte, that is baseline resolved. A chromatography experiment resulting in two or more distinct peaks refers to a chromatogram in which a first baseline resolved distinct peak is resolvable from a second baseline resolved distinct peak. The methods of the present disclosure may allow for the resolution of two or more distinct peaks in a chromatogram, each distinct peak corresponding to a unique detected analyte.

The separation of nucleic acids using the described methods can be achieved with high resolution by manipulating, for example, the particle size, column length, and column pressure. By adjusting said variables according to the teachings described herein and as shown in the examples (see, e.g., Example 4), maximum resolution can be achieved for DNA varying in nucleotide length.

### BRIEF DESCRIPTION OF THE DRAWINGS

FIGS. 1A-1C depict a liquid chromatography system and flow path for performing direct affinity-slalom according to embodiments of the technology.

FIG. 1A shows the flow path across an LC system set up according to embodiments of the technology.

FIG. 1B provides an enlarged view of the zero dead volume or low volume union between an affinity chromatography column and a slalom column.

FIG. 1C provides an exemplary zero dead volume union.

FIG. 2A shows a gel electrophoresis experiment performed on an exemplary ssRNA ladder.

FIG. 2B shows a gel electrophoresis experiment performed on an exemplary dsRNA sample (right hand lane). The left hand lane is an ssDNA ladder.

FIG. 3 shows a chromatogram resulting from a slalom chromatography experiment separating a sample including a mixture of ssRNA ladder and a dsRNA sample. The ssRNA ladder and the dsRNA sample were mixed in a 50:50 ratio (solid line), a 10:90 ratio (dotted line), a 5:95 ratio (dot-dash line), and a 1:99 ratio (dashed line).

FIG. 4A shows a chromatogram resulting from the sample injection of a direct affinity-slalom chromatography experiment separating a sample including a mixture of ssRNA ladder and a dsRNA sample. The ssRNA ladder and the dsRNA sample were mixed in a 50:50 ratio (solid line), a 10:90 ratio (dotted line), a 5:95 ratio (dot-dash line), and a 1:99 ratio (dashed line)

FIG. 4B shows a chromatogram resulting from the injection breakthrough of a direct affinity-slalom chromatography experiment separating a sample including a mixture of ssRNA ladder and a dsRNA sample. The ssRNA ladder and the dsRNA sample were mixed in a 50:50 ratio (solid line), a 10:90 ratio (dotted line), a 5:95 ratio (dot-dash line), and a 1:99 ratio (dashed line)

FIG. 5 shows a chromatogram resulting from the eluent of a direct affinity-slalom chromatography experiment wherein no wait time is provided between the sample injection and the elution injection. The sample included a mixture of ssRNA ladder and a dsRNA sample. The ssRNA ladder and the dsRNA sample were mixed in a 50:50 ratio (solid line), a 10:90 ratio (dotted line), a 5:95 ratio (dot-dash line), and a 1:99 ratio (dashed line).

### DETAILED DESCRIPTION

Disclosed herein are methods of performing tandem affinity chromatography and slalom chromatography using a direct elution method from the affinity chromatography column to the slalom column (direct affinity-slalom chromatography; or alternatively direct affinity-slalom).

Slalom chromatography is a powerful alternative to conventional electrophoretic methods. Conventional electrophoretic approaches can be time intensive (ranging from multiple hours to days) and are not sensitive enough to detect nucleic acid heterogeneity, and hinder downstream processing of samples due to the need to extract nucleic acids from the gel itself, substantially lowering yield and increased contaminants. Slalom chromatography, meanwhile, utilizes high shear, high flow conditions, allowing for rapid separation of nucleic acid species.

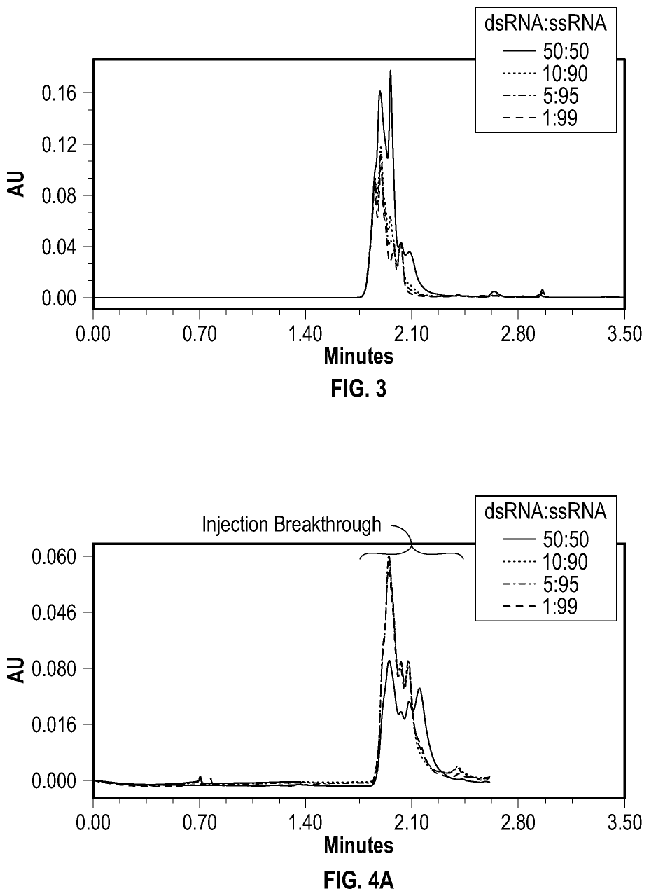

Although high-flow conditions are well suited for separating varying sizes of a single nucleic acid type, slalom chromatography may struggle to separate more complex mixtures of nucleic acids (e.g., mixtures containing multiple classes of oligonucleotides; wherein the mixture includes multiple nucleic acid sizes of each class of oligonucleotide). This difficulty is highlighted in exemplary FIG. 3, which shows the attempted slalom chromatography separation of a mixture including ssRNA oligonucleotides of a plurality of lengths and dsRNA of a plurality of lengths. As highlighted in the exemplary data, although good separation is observed for each of the ssRNA molecules from each other ssRNA molecule, and good separation is observed for each dsRNA molecule from each other dsRNA molecule, the elution of the ssRNA and the dsRNA overlap.

The present technology relates to methods of performing high-flow chromatography under adiabatic conditions. The disclosed methods are capable of separating nucleic acids by size in under 10 minutes with high efficiency.

In conventional methods, the use of different modes of separation (i.e., affinity chromatography and slalom chromatography) on a single sample requires the use of either valve switching or sample isolation from the first mode of separation (also known as a dimension). The former requires liquid chromatography systems with valve and trap configurations that can be used to first capture (‘trap’) an oligonucleotide of interest separated by a first dimension and divert its flow path to the second mode of separation. In these configurations, the modes of separation (dimensions) are decoupled—that is only the trapped oligonucleotides from the first dimension are transferred to and separated by the second dimension. Thus, the ability to separate multiple target oligonucleotides is limited, in part, by the system configuration and the efficiency of the valve/trap apparatuses. An alternative to valving is to first collect a sample fraction from a first mode of separation, and subsequently injecting the sample fraction onto the second mode of separation. This approach requires manual manipulation of small sample volumes, which can complicate workflows and introduce user error. These approaches are necessary as direct connection of one column type to another in the absence of a valve would result in either many peaks or single broad peaks being eluted from the first column into the second column, thereby reducing separation performance of the second column.

The direct affinity-slalom methods described herein circumvent the aforementioned issues with two-dimensional chromatography while permitting separation of a sample using two chromatographic modes of separation.

In particular, the affinity chromatography columns described herein result in a single load peak from the load step (i.e., a single peak of oligonucleotides, wherein the single peak of oligonucleotides is composed of one or more oligonucleotides that do not bind to the affinity column) and a single, elution peak from the elution step (i.e., a single peak of oligonucleotides, wherein the single peak of oligonucleotides is composed of one or more oligonucleotides), which is on the order of a standard HPLC/UHPLC injection (i.e., between 5-20 μL). Further, the affinity chromatography columns used in the instant technology have pressure compatibility with the slalom columns used herein. Thus, the peaks (the one or more oligonucleotides which elute together in either the load step or the elution step) that elute from the affinity chromatography column can be eluted directly onto the slalom column without the need for valving or fraction collection. In doing so, both the oligonucleotides from the loading step and the elution step are subsequently separated by the slalom column.

Affinity chromatography and slalom chromatography are both adsorptive methods of chromatography (i.e., one or more target oligonucleotides are retained by the chromatography column until exposed to a buffer condition which causes the release of the one or more oligonucleotides). Therefore, methods of the present disclosure allow for a first group of oligonucleotides to be captured in the affinity chromatography column during a first affinity chromatography step and retained during an injection breakthrough in the slalom chromatography column and/or a wait time. Then, during an elution step, the first group of oligonucleotides may be eluted from the affinity chromatography column onto the slalom chromatography column, allowing the target oligonucleotide to be focused within the slalom chromatography column.

In preferred embodiments, the one or more target oligonucleotides include a single stranded RNA (ssRNA, e.g., mRNA). Methods of the present disclosure are particularly well suited to separating a target oligonucleotide including ssRNA from a sample including the target oligonucleotide and one or more double stranded RNA (dsRNA).

### Affinity Chromatography Materials and Columns Thereof

The present technology utilizes affinity chromatography columns for the purification and isolation of oligonucleotides. The affinity chromatography columns are suitable for use in a high-performance liquid chromatography (HPLC) system or an ultra-high performance liquid chromatography (UHPLC) system and are designed for robust on-column affinity capture at the high pressures and flow conditions of said systems.

### Affinity Chromatography

A sample to be separated by a method disclosed herein may first be separated by an affinity chromatography column. Affinity chromatography serves to separate one or more components of a sample based on the strength of interaction between the component and an affinity agent included with the affinity chromatography column. The strength of these interactions can be controlled by, e.g., the pH of the mobile phase (i.e., the pH of the buffer). Affinity chromatography columns of the present disclosure are suitable for use in a high-performance liquid chromatography (HPLC) system or an ultra-high performance liquid chromatography (UHPLC) system and are designed for robust on-column affinity capture at the high pressures and flow conditions of said systems.

### Affinity Chromatography Materials

The affinity chromatography columns used in the methods disclosed herein include particles which provide high surface area for conjugation of affinity agents and can withstand the pressures of HPLC and UHPLC systems. In some embodiments, the affinity chromatography column includes a plurality of nonporous polymer particles. In some embodiments, the plurality of nonporous polymer particles having an average particle size between 1.0 μm to 10 μm. In some embodiments, each particle within the plurality of particles is highly spherical with a smooth surface. In some embodiments, each particle within the plurality of particles is highly spherical with a bumpy, convex surface. Such materials have surface areas (measured in m²/g) that are close to their theoretical values. The theoretical surface area for a nonporous smooth sphere is equal to 6/{particle diameter×particle density}. For example, 1 μm polymer particles with a density of approximately 1 g/mL has a theoretical surface area of 6 m²/g, a 3.5 μm polymer particle with the same density has a theoretical surface area of 1.7 m²/g, and a 7 μm polymer particle with same density has a theoretical surface area of 0.9 m²/g.

The particles for use in the methods described herein are nonporous. While some pores or porosity may be incorporated within the particles as discontinuities or as microporosity, nonporous particles are those particles having a pore volume that is less than 0.1 cc/g of the material forming the particle. Preferably, nonporous particles have a pore volume that is less than 0.10 cc/g (e.g., 0.05 cc/g), and preferably less than 0.02 cc/g, in some embodiments. Pore volume is determined using methods known in the art based on multipoint nitrogen sorption experiments (Micromeritics ASAP 2400; Micromeritics Instruments Inc., Norcross, GA). Without wishing to be bound by theory, it is believed that the use of nonporous particles is advantageous as it removes diffusion of oligonucleotides into pores of the particles, thereby by improving kinetics of the binding and eluting steps of affinity chromatography.

The nonporous particles described herein have an average particle size of between 1 to 10 μm. In some embodiments, the particle size is about 1.7 μm. In some embodiments, the particle size is about 3.5 μm. In some embodiments, the particle size is about 7 μm.

The size (e.g., less than 10 microns), shape (e.g., spherical), and surface area (e.g., nonporous smooth or nonporous bumpy convex) create a form factor useful for affinity chromatography and affinity chromatography columns used in conjunction with HPLC and UHPLC systems. These systems operate under high pressures (e.g., typically greater than 3,000 psi, such as, for example, 5,000 psi, 10,000 psi, 12,000 psi, 15,000 psi and so forth). Therefore, the particles used herein are rigid particles such that the form factor is retained under HPLC and UHPLC operating conditions.

As used herein, the term “rigid particle,” as used herein, refers to the strength of the particle to withstand applied pressures under flow conditions. A rigid particle appears visually undamaged (i.e., maintains the same form factor without breaking, crushing, or alteration) in a scanning electron microscope image after exposure to pressures of 3,500 psi, wherein less than 10% of the observed particles are visually damaged. In addition, particles in a packed bed that are broken or deformed result in reduced flow and increased pressure as one would predict using the Kozeny-Carmen equation. Broken or deformed particles in a packed bed can increase pressure beyond levels suitable for use in HPLC or UHPLC.

Materials that meet the form factor requirements for forming a core (e.g., center or base) of the particles used herein include polymers, in particular organic polymers. Thus, in some embodiments, the nonporous particles include a nonporous polymer core. In some embodiments, the nonporous polymer core is divinylbenzene (DVB), for example divinylbenzene 80%. In some embodiments, the nonporous polymer core is formed to include two or more polymers. For example, in some embodiments the nonporous polymer cores include both DVB and polystyrene. In certain embodiments, the nonporous polymer cores can be manufactured to include a gradient in the polymer composition. For example, the inner portion of the core can be formed of 100% of first polymer (i.e., polymer A) and an outer portion of the core can be formed of 100% or some percentage greater than 0% of a second polymer (i.e., polymer B). Radially from the inner portion to the outer portion of the core, the percentage of polymer A and polymer B can vary to form the gradient in polymer composition. Other embodiments of nonporous polymer cores and particles suitable for use with the present technology are described in U.S. Patent Publication No. 2019/0322783, incorporated herein by reference.

Other nonporous materials can be utilized as long as the form factor of the particles can be maintained under the operating conditions of HPLC or UHPLC. That is, other materials, such as silica, metal oxides, hybrid inorganic-organic materials, or combinations thereof may be used to create nonporous spherical particles having an average particle size of less than 10 microns and the rigidity to retain form factor under high operating pressures (e.g., greater than 3000 psi).

### Affinity Agents

To form particles useful for affinity chromatography, the outer surface of the affinity chromatography particle is conjugated, either directly or indirectly, to an affinity agent. The affinity agent is configured to interact (and therefore bind) with the first group of oligonucleotides, and not interact with the second group of oligonucleotides. This allows the affinity chromatography column to retain the first group of oligonucleotides while the second group of oligonucleotides pass from the affinity chromatography column to the slalom chromatography column during the injection step. The first group of oligonucleotides can then be released from the affinity chromatography column, e.g., with an elution injection.

The affinity agent may be bound directly or indirectly to the surface of the affinity chromatography particle. In embodiments for direct conjugation, the outer surface of the nonporous polymer core includes a hydrophilic surface, such as, for example, an epoxide. One or more affinity agents can be directly conjugated to the hydrophilic surface. The hydrophilic surface (or hydrophilic layer, used interchangeably herein) can be formed of a polymer, molecule or siloxane that has a high density of hydrophilic groups (e.g., hydroxyls, PEG, sugars or carbohydrates). The immobilization of these hydrophilic groups can occur by condensation (ester, amid, silanol, silyl ether), polymerization (methacrylates, acrylates, styryl) epoxy activation (epihydrochlorin), or ether formation (direct attachment of PEG or carbohydrate groups by ether formation). Further examples include (3-glycidyloxypropyl)trimethoxysilane, (3-glycidyloxypropyl)triethoxysilane, polyacrylate, poly(methyl acrylate), and combinations thereof. Additionally or alternatively, these may include glycidol, glyceroltriglycidyl ether, and combinations thereof.

An affinity agent can be directly conjugated to the hydrophilic surface of the nonporous particle via linkers and methods known in the art, and described in Hermanson G, “Bioconjugate Techniques” 3^{rd} Edition, July 2013).

In embodiments for indirect conjugation, one or more streptavidin molecules are first directly conjugated to the hydrophilic surface of the nonporous particles as described above. Due to the strong affinity between biotin and streptavidin, the streptavidin molecules provide a binding site for biotinylated affinity agents, providing a functionalized particle with a specific affinity (based on the affinity of the affinity agent).

In preferred embodiments, the affinity agent is configured to bind to an oligonucleotide. In some embodiments, the affinity agent is an oligonucleotide, nucleic acid, or oligomer. In some embodiments, the oligonucleotide can range from 5-50 nucleotides. Any or all of the nucleotides in a particular oligonucleotide or nucleic acid species can further be modified using methods known in the art, including biotinylating of the oligonucleotide or nucleic acid species. In particular, an oligonucleotide can be biotinylated on the 5′ or 3′ end.

Methods of the present disclosure include nucleic acid affinity agents and oligonucleotide affinity agents. The oligonucleotide may include deoxyribonucleic acids (DNA), ribonucleic acids (RNA), or a combination thereof. DNA oligonucleotides include the nucleotides cytidine, guanosine, adenosine, and thymidine. RNA oligonucleotides include the nucleotides cytidine, guanosine, adenosine, and uridine. In some embodiments, the oligonucleotide may include nucleic acid analogues (i.e., non-naturally occurring nucleic acids or analogues thereof). Examples of nucleic acid analogues include peptide nucleic acids, locked nucleic acids, glycol nucleic acids, threose nucleic acids, hexitol nucleic acids. Nucleic acid analogues are further reviewed in Wang et al., Molecules (2023) 28(20):7043; the nucleic acid analogues of which are incorporated herein by reference. Oligonucleotides may further be modified at the nucleobase, sugar, or phosphodiester backbone with an array of chemical modifications which are further reviewed in Epple et al., Emerg. Top. Life. Sci. (2021) 5(5):691-697; the oligonucleotide modifications of which are incorporated herein by reference. For example, the oligonucleotide modifications may include a phosphorothioate, a 2′ methoxide, or a 2′ fluoride. In yet other embodiments, the nucleic acid affinity agent is an aptamer having specificity to a target oligonucleotide, including a biotinylated aptamer. Methods of biotinylating oligonucleotides, including modified oligonucleotides or oligonucleotides including non-naturally occurring nucleic acid analogues, are well known in the art and would be readily understood by a person of ordinary skill.

Oligonucleotides suitable as affinity agents may be presented by the following Formula I:

B₀₋₁(Xₙ)ₚB′₀₋₁ (Formula I);
wherein B and B′ are independently a biotin group,
X is independently a nucleotide, nucleoside, or derivative thereof, including but not limited to adenosine, thymidine, guanosine, and cytidine;
n is independently 1-50; and
p is independently 1-50.

In some embodiments of Formula I, at least one of B or B′ is present (i.e., B or B′ is 1). In some embodiments, B is 1 and B′ is 0. In some embodiments, B is 0 and B′ is 1. In some embodiments, both B and B′ are 1. In some embodiments, both B and B′ are 0.

In some embodiments of Formula I, B is 1, X is thymidine, n is 25, p is 1, and B′ is 0. In some embodiments of Formula I, B is 0, X is thymidine, n is 25, p is 1, and B′ is 1. The resultant 5′-biotinylated or 3′-biotinylated oligonucleotide includes 25 thymidine units (i.e., a 25-mer of thymidine or dT₂₅).

In some embodiments, the affinity agent is a biotinylated oligonucleotide of Formula I. In some embodiments, the biotinylated oligonucleotide sequence is complementary to a target oligonucleotide sequence.

In some embodiments, the affinity agent is an antibody (e.g., a nanobody or a monobody) which specifically binds to a type of oligonucleotides. For example, the affinity agent may be the anti-dsRNA J2 monobody. The J2 monobody specifically binds to dsRNA, allowing oligonucleotides which are not dsRNA (e.g., ssRNA) to pass through the affinity chromatography column to the slalom chromatography column in loading step. The dsRNA may then be released from the affinity chromatography column with an elution injection. Other exemplary antibody affinity agents include the K1 monobody.

### Affinity Chromatography Column Body

The affinity chromatography materials, i.e., the nonporous particles conjugated to an affinity agent, are typically packed into a chromatographic column, thereby resulting in an affinity chromatography column. The column body is typically formed of a metal or a metal alloy, e.g., titanium or stainless steel.

In some embodiments, an alkylsilyl coating or other high-performance surface (HPS) is provided to limit or reduce non-specific binding of a sample with the walls or interior surfaces of the column body. Without wishing to be bound by any particular theory, it is believed than an alkylsilyl coating covering metal surfaces prevents or otherwise minimizes contact between fluids passing through the column. The alkylsilyl coating can be applied to the interior surfaces defining what is known as a wetted path of the column. A metal wetted path includes all surfaces formed from metal that are exposed to fluids during operation of the chromatographic column. The metal wetted path includes not only the column body walls but also metal frits disposed within the column. The coating may be applied not only to the wall of the column body but also to the frits.

In general, the alkylsilyl coating is applied through a vapor deposition technique. Vaporized precursors are charged into a reactor in which the part to be coated is located. These vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating is applied to other portions of the liquid chromatography system. For example, the alkylsilyl coating can be applied to metal components residing upstream and downstream of the column. Specifically, the alkylsilyl coating can be applied to an injector of the liquid chromatography system and to post column tubing and connectors (e.g., tubing and connectors leading from the column to downstream components such as detectors). Further, the affinity chromatographic columns of the present technology do not require the addition of additional organic modifiers to reduce non-specific binding. Typically, the addition of an organic modifier (e.g., acetonitrile) may be necessary with sorbents used in affinity chromatography to reduce non-specific binding. Due to the already low non-specific binding of the columns of the present technology, no organic modifier is necessary.

In one embodiment, the alkylsilyl coating includes a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. Other embodiments of alkylsilyl coatings suitable for use with the present technology are described in US Patent Publication No. 2019/0086371 and US Application Publication No. 2022/0118443.

### Slalom Chromatography

A sample to be separated by a method disclosed herein may be eluted from an affinity chromatography column to a slalom chromatography column, e.g., by a zero dead volume or a low dead volume union. Slalom chromatography is a powerful alternative to conventional electrophoretic methods. The conventional electrophoretic approaches can be time intensive (ranging from multiple hours to days) and are not sensitive enough to detect nucleic acid heterogeneity, and hinder downstream processing of samples due to the need to extract nucleic acids from the gel itself, substantially lowering yield and increased contaminants. Slalom chromatography, meanwhile, utilizes high shear, high flow conditions, allowing for rapid separation of nucleic acid species.

Although high-flow conditions are well suited for separating varying sizes of a single nucleic acid type, slalom chromatography may struggle to separate more complex mixtures of nucleic acids (e.g., mixtures containing multiple classes of oligonucleotides; wherein the mixture includes multiple nucleic acid sizes of each class of oligonucleotide). This difficulty is highlighted in exemplary FIG. 3, which shows the attempted slalom chromatography separation of a mixture including ssRNA oligonucleotides of a plurality of lengths and dsRNA of a plurality of lengths. As highlighted in the exemplary data, although good separation is observed for each of the ssRNA molecules from each other ssRNA molecule, and good separation is observed for each dsRNA molecule from each other dsRNA molecule, the elution of the ssRNA and the dsRNA overlap.

The present disclosure relates to methods of performing high-flow chromatography under adiabatic conditions. The disclosed methods are capable of separating nucleic acids by size in under 10 minutes with high efficiency.

### High-Flow Chromatography

The methods disclosed herein utilize conditions that result in a high shear rate such that the nucleic acid molecules in the column are extended/elongated under the shear flow. Extension or elongation of nucleic acid molecules can be determined by the Weissenberg number, which compares elastic and viscous forces exerted on a molecule. The Weissenberg number is a dimensionless metric that is a function of relaxation time and shear rate:

Wi=τ_{R}\dot{γ}
wherein τ_{R} is the relaxation time (seconds) as represented by the formula:

$${τ}_{R} = \frac{{nL}_{c}^{2} {I}_{P}}{{k}_{B} T}$$

wherein h is dynamic velocity (Pa*s); L_{C} is contour length (m); lₚ is persistence length (m); k_{B} is the Boltzmann constant (1.38×10⁻²³ J·K⁻¹), and T is temperature (Kelvin); and
   \dot{γ} is shear rate (seconds⁻¹) as represented by the formula:

$$\dot{γ} = \frac{Δ u}{Δ z} = \frac{16}{3} 〈 u 〉 / d$$

wherein Δu is the velocity difference between two points in a direction perpendicular to the flow direction, the two points separated by a distance of Δz; <u> is the average linear interstitial velocity along the packed bed; and
d is the average flow-through tube diameter.
   The relaxation time τ_{R} is dependent, in part, on intrinsic properties of the target nucleic acid. In certain instances, the relaxation time of a molecule under certain conditions is known in the art. For example, the L_{C} of linear dsDNA is equal to the number of base pairs*3.4 Å; the dynamic velocity of linear dsDNA is 1.2 cP; and the persistence length of linear dsDNA is 450 Å. Alternatively, relaxation time of a molecule under certain conditions may be determined empirically using known methods that are readily understood by one of ordinary skill in the art. (see e.g., Bouchiat et al., Biophysical Journal (1999) 76:409-413).

In instances wherein the Weissenberg number is greater than 1, the nucleic acid molecule is extensible under the shear flow and would be separated by size based on the principles of slalom chromatography. In instances wherein the Weissenberg number is less than 1, the nucleic acid molecule is not extensible under the shear flow and would not be separated by size based on the principles of slalom chromatography.

Accordingly, in one aspect the methods disclosed herein utilize conditions that result in the nucleic acid molecules exhibiting a Weissenberg number (Wi) of greater than 1. In some embodiments, the Wi is between 1-100, 5-100, 10-100, 15-100, 20-100, 25-100, 30-100, 35-100, 40-100, 45-100, 55-100, 60-100, 65-100, 70-100 75-100, 80-100, 85-100, 90-100, or 95-100.

Shear rate, and consequently the Weissenberg number (Wi), can be influenced by several parameters, including flow rate, particle size, mobile phase viscosity, and temperature. Thus, said variables may be adjusted to achieve sufficient Wi numbers for the separation of the desired nucleic acid molecules.

The choice of column and particle, particularly with respect to column length and particle size, is in part dependent on the size of the nucleic acid molecules to be separated and detected as would be appreciated and readily understood by one of ordinary skill in the art. In some embodiments, the column and particle are chosen such that the chromatography experiment produces a desired zone retention factor (k₁). In some embodiments, the desired zone retention factor is less than 0.35. In some embodiments, the desired zone retention factor is from about −0.25 to 0.35. In some embodiments, the desired zone-retention factor is about 0. In some embodiments, the column and particle are chosen such that the elution of a desired analyte from the column can be completed in a desired time. In some embodiments, the desired time is from about 0.6 minutes to about 10 minutes. In some embodiments, the desired time is from about 1 minute to about 3 minutes.

A chromatography system disclosed herein may further include one or temperature sensors, each temperature sensor configured to sense a temperature along a different portion of a length of the chromatography column (e.g., a temperature sensor configured to sense the temperature of the inlet region of the liquid chromatography column, a temperature sensor configured to sense the temperature of the outlet region of the liquid chromatography column, or a temperature sensor configured to sense the temperature of a middle region between the inlet region and the outlet region). In some embodiments, the chromatography system further includes a plurality of temperature sensors (e.g., at least 2 temperature sensors, at least 3 temperature sensors, at least 4 temperature sensors, at least 5 temperature sensors, at least 10 temperature sensors, at least 15 temperature sensors, at least 20 temperature sensors, at least 30 temperature sensors, etc.). In some embodiments, the plurality of temperature sensors includes at least an inlet temperature sensor configured to determine the temperature of the inlet region and an outlet temperature sensor configured to determine the temperature of the outlet region. In some embodiments, the chromatography system further comprises at least one processor operative to execute the computer-executable code to cause the apparatus to determine the temperature of the liquid chromatography column from the plurality of temperature sensors, and individual control at least one of the temperature sensors based on the inlet temperature or the outlet temperature. In some embodiments, the processor may communicate with one or more openings in the vacuum chamber. In some embodiments, one of the one or more openings is in fluid communication with a vacuum pump. In some embodiments, one of the one or more openings is in fluid communication with a source of inert gas. By modulating communication with a vacuum pump and a source of inert gas, the processor may control the thermal conductivity of the vacuum chamber.

### Slalom Chromatography Materials

A slalom chromatography column may include a slalom chromatography material. Without wishing to be bound by theory, it is understood that multiple chromatographic principles may influence retention of an analyte (e.g., a DNA or RNA molecule) based on the size and relaxation time of the analyte. For example, under increased flow rate (i.e., high shear rates), analytes with longer relaxation times will have increased retention on a slalom chromatography column. Described herein are exemplary materials which may be used in a slalom chromatography column.

In some embodiments, the slalom chromatography material includes a plurality of slalom chromatography particles. The plurality of slalom chromatography particles may include an average diameter from about 1 μm to about 10.5 μm. In some embodiments, the plurality of slalom chromatography particles include a diameter of from about 1 μm to about 2 μm, from about 2 μm to about 3 μm, from about 3 μm to about 4 μm, from about 4 μm to about 5 μm, from about 5 μm to about 6 μm, from about 6 μm to about 7 μm, from about 7 μm to about 8 μm, from about 8 μm to about 9 μm, from about 9 μm to about 10 μm, or from about 10 μm to about 10.5 μm. In some embodiments, the plurality of slalom chromatography particles includes a diameter from about 1.5 μm to about 1.9 μm. In some embodiments, the plurality of slalom chromatography particles includes a diameter from about 2.8 μm to about 3.2 μm. In some embodiments, the plurality of slalom chromatography particles includes a diameter of from about 4.8 μm to about 5.2 μm. In some embodiments, the plurality of slalom chromatography particles includes a diameter of from about 9.8 μm to about 10.2 μm. In some embodiments, the plurality of slalom chromatography particles includes a diameter of about 1.7 μm. In some embodiments, the plurality of slalom chromatography particles includes a diameter of about 3.0 μm. In some embodiments, the plurality of slalom chromatography particles includes a diameter of about 5.0 μm. In some embodiments, the plurality of slalom chromatography particles includes a diameter of about 10 μm.

In some embodiments, the plurality slalom chromatography particles include a negatively charged surface, which contributes to electrostatic repulsion of the negatively charged nucleic acid molecules and the particle surface. Without wishing to be bound by any particular theory, distance of the nucleic acid molecules from the particle surface sharpens peak widths and increases separation speed at a constant resolution. Said electrostatic repulsion can further be adjusted by altering the ionic strength of the mobile phase, e.g., by increasing or decreasing the salt content of the mobile phase.

In some embodiments, the plurality of slalom chromatography particles are nonporous slalom chromatography particles. In some embodiments, the plurality of slalom chromatography particles are porous slalom chromatography particles. In some embodiments, the porous slalom chromatography particles have an average pore diameter of between 20 Å to 900 Å. In some embodiments, the average specific pore volume of the plurality of particles is from about 0.5 cm³/g to about 1.3 cm³/g. Without wishing to be bound by any particular theory, increasing the pore size may shift the retention of larger molecules relative to the smaller nucleic acid species.

In some embodiments, the plurality of slalom chromatography particles may include an ordered structure. The ordered structure may be described using space groups, or a crystallographic group equivalent thereto. Exemplary space groups include the simple cubic space group (alternatively referred to as cP, Pm3̅m, or space group number 221), the body-centered cubic space group (alternatively referred to as bcc, Im3m, or space group number 229), or the hexagonal closest packing space group (alternatively referred to as hcp, P63/mmc, or space group number 194). In some embodiments, the particles are packed in a hexagonal closest packing space group.

In some embodiments, the slalom chromatography column may include an ordered material. In the context of chromatography materials, having an ordered structure refers to material with an internal structure which can be structurally defined (e.g., through a crystallographic space group), an internal structure which is periodic (e.g., periodic along one axis, periodic along two axes, or periodic along three axes), or that has been designed by a manufacturer (e.g., a material prepared through additive manufacturing). A structure may be ordered in two dimensions (e.g., a two-dimensional array with a defined spacing between each member of the array) or in three dimensions (e.g., a plurality of packed particles). The structure of a material including an ordered structure may be described either through the position of material within the structure, or the location of void space (i.e., space through which a mobile phase and/or an analyte may flow during a chromatography experiment) within a material.

In some embodiments, the ordered structure includes a two dimensional array of microstructures (e.g., pillars) arranged on a surface. Each microstructure of the microstructure array may be oriented transverse (e.g., perpendicular) to the flow path of the chromatography column such that an analyte passes through the void space between a plurality of pillars of the 2D array as the analyte traverses the column. Each microstructure may be further modified to change the interaction between the microstructure and an analyte. For example, the microstructure may include a plurality of ridges, thereby increasing the total surface area of the microstructure which may interact with the analyte. A microstructure array may further include any number of components which direct the flow path of an analyte. For example, a microstructure array may include a collector, a distributor, or a plurality of turns. A collector may be used to collect the narrowest band of a sample (i.e., the band of sample which elutes over the narrowest range of time) after separation. A distributor may be used to inject a sample at an even volume across the chromatography material. Turns may be used to minimize the dispersion of an analyte during a chromatography experiment. Turns may be specifically useful in chromatography columns including longer column lengths.

In some embodiments, the ordered structure may include a plurality of cylindrical pillars on a surface. In some embodiments, each pillar of the micropillar array may have a length of from about 0.1 μm to about 100 μm. In some embodiments, each pillar of the micropillar array may have a length of from about 0.5 μm to about 25 μm. In some embodiments, each pillar of the micropillar array may have a width of from about 0.5 μm to about 10 μm. In some embodiments, each pillar of the micropillar array is separated from its nearest neighbor or (in the case where a pillar is equidistant from more than one other pillars) nearest neighbors by a distance of from about 0.5 μm to about 10 μm.

In some embodiments, a pillar array may be described by its external porosity (&e). Similar to the porosity of a conventional bulk material, external porosity creates additional and/or alternative flow paths through a chromatography material, thereby influencing the flow rate of the mobile phase through the material, and the resulting separation of analytes by the material. It will be understood that a specific size of analyte (e.g., an oligonucleotide consisting of a specific number of base pairs) may be best separated by a pillar array of a specific external porosity, a specific pillar radius, a specific separation between adjacent pillars, a specific pillar height, or a specific distribution of pillar diameters.

In some embodiments, the slalom chromatography material includes a three-dimensional material with a defined structure (e.g., a channel or a flow path) running through the material. For example, the slalom chromatography material may include a gyroid.

Slalom chromatography materials may be composed of inorganic materials, organic materials, or inorganic/organic hybrid materials. In some embodiments the slalom chromatography material is a plurality of inorganic slalom chromatography particles, a plurality of organic slalom chromatography particles, or a plurality of inorganic/organic hybrid slalom chromatography particles. Exemplary slalom chromatography materials, but are not limited to, ethylene bridged hybrid particles (BEH; comprising tetraethoxysilane (TEOS) and bis(triethoxysilyl) ethane as described in U.S. Pat. Nos. 6,686,035, and 7,250,214, incorporated herein by reference. Additional slalom chromatography particle compositions include, for example, hybrid inorganic/organic particles such as those described in U.S. Pat. Nos. 11,291,974 and 9,145,481. Additional slalom chromatography particles may include polystyrene, divinylbenzene, polyacrylamides, and polymethylacrylates with varying degrees of crosslinking.

### Slalom Chromatography Column Body

The slalom chromatography column body of a slalom chromatography column described herein may be characterized by an inner diameter, a length, and a material composition. In some embodiments, the inner diameter of the slalom chromatography column body is from about 2.1 mm to about 7.8 mm. In some embodiments, the slalom chromatography column body length may be from about 10 mm to about 300 mm. Exemplary column dimensions include, but are not limited to, 2.1×20 mm, 2.1×50 mm, 2.1×100 mm, 2.1×150 mm, 4.6×50 mm, 4.6×100 mm, 4.6×150 mm, and 4.6×300 mm. Exemplary slalom chromatography column body materials include stainless steel, polyetheretherketone (PEEK) lined steel, titanium, or a stainless alloy.

In some embodiments, an interior surface of the slalom chromatography column body is treated to reduce non-specific binding and enhance overall efficiency of the chromatographic system. In particular, an alkylsilyl coating or other high performance surface is provided to limit or reduce non-specific binding of a sample with walls or interior surfaces of a slalom chromatography column body. Without wishing to be bound by theory, it is believed that an alkylsilyl coating covering metal surfaces prevents or minimizes contact between fluids passing through the slalom chromatography column body and the interior surfaces of the column. Typically, the alkylsilyl coating is applied to metal surfaces defining what is known as a wetted path of the column. A metal wetted path includes all surfaces formed from metal that are exposed to fluids during operation of the chromatographic column. The metal wetted path includes not only slalom chromatography column body walls but also metal frits disposed within the column.

In general, the alkylsilyl coating is applied through a vapor deposition technique. Precursors are charged into a reactor in which the part to be coated is located. Vaporized precursors react on the surfaces of the part to be coated to form a first layer of deposited material. The vapor deposition can be applied in a stepwise function to apply a number of layers of deposited material to the surfaces to grow a thickness of the coating and/or to apply layers of different materials (e.g., alternating between a first and second material) to form the coating.

In some embodiments, the alkylsilyl coating comprises a hydrophilic, non-ionic layer of polyethylene glycol silane. In another embodiment, the alkylsilyl coating is formed from one or more of the following precursor materials bis(trichlorosilyl) ethane or bis(trimethoxysilyl) ethane. In some embodiments, the high-performance surface is a C2-PEG. A C2-PEG coating may be prepared as described in U.S. Publication No. 2022/0118443. For example, an organosilane precursor, such as bis(trichlorosilyl) ethane or bis(trimethoxysilyl) ethane, may first be vapor deposited on a metal surface, including an interior surface of the column or a metal frit of the column. Following vapor deposition of the organosilane precursor, the coated metal components may be treated with a toluene solution of a polyethylene glycol (PEG), for example, 2-[methoxy (polyethyleneoxy)₆₋₉propyl]tris(dimethylaminosilane). The reagent solution may be reacted with the metal components for ~3 days, washed in toluene, washed in isopropanol, and vacuum dried at 70 °C, resulting in a C2-PEG coating. Other embodiments of alkylsilyl coatings suitable for use with the present technology are described in U.S. Patent Publication No. 2019/0086371 and U.S. Application Publication No. 2022/0118443 (which are hereby incorporated by reference; the coatings of which are incorporated herein by reference).

### Oligonucleotide Analytes

The methods disclosed herein may be used for the separation of analytes based on their elongation under high-flow conditions. In preferred embodiments, methods of the present disclosure relate to the separation of nucleic acids, including DNA, RNA, or mixtures thereof.

In some embodiments, methods of the present disclosure relate to methods of separating a plurality of oligonucleotides. For example, a method of the present disclosure may include separating a sample, the sample including a plurality of oligonucleotides, into a first group of oligonucleotides and a second group of oligonucleotides. In some embodiments, the first group of oligonucleotides includes a ribonucleic acid and the second group of oligonucleotides includes a deoxyribonucleic acid. In some embodiments, the first group of oligonucleotides includes a single stranded oligonucleotide and the second group of oligonucleotides includes a double stranded oligonucleotide. In some embodiments, the first group of oligonucleotides includes a single stranded oligonucleotide and the second group of oligonucleotides includes a double stranded oligonucleotide. In some embodiments, the first group of oligonucleotides includes ssRNA and the second group of oligonucleotides includes dsRNA. In preferred embodiments, methods of the present disclosure separate the first group of oligonucleotides from the second group of oligonucleotides using affinity chromatography in an affinity chromatography column.

A method may further include separating oligonucleotides of the first group and/or oligonucleotides of the second group using the principles of high-flow chromatography (i.e., slalom chromatography). For example, a method of the present disclosure may first separate a sample into a first group of oligonucleotides (e.g., including ssRNA) and a second group of oligonucleotides (e.g., including dsRNA) using affinity chromatography. The breakthrough from the affinity chromatography column (e.g., the second group of oligonucleotides) may then be eluted onto a slalom chromatography column, enabling the further separation of oligonucleotides by slalom chromatography (e.g., further separating the second group of oligonucleotides by size). Similarly, the eluent from the affinity chromatography column may then be separated by slalom chromatography.

In some embodiments, the plurality of oligonucleotides includes a plurality of single-stranded DNA (ssDNA). In some embodiments, the plurality of oligonucleotides includes a plurality of double-stranded DNA (dsDNA). In some embodiments, the plurality of oligonucleotides includes a plurality of RNA. In some embodiments, the plurality of oligonucleotides includes a plurality of double-stranded RNA (dsRNA). In some embodiments, the plurality of oligonucleotides includes a plurality of a first oligonucleotide (e.g., DNA (e.g., ssDNA or dsDNA) or RNA (e.g., ssRNA or dsRNA)) and a plurality of a second oligonucleotide (e.g., DNA (e.g., ssDNA or dsDNA) or RNA (e.g., ssRNA or dsRNA)).

In some embodiments, a method described herein may be used to separate a plurality of DNA and a plurality of RNA. In some embodiments, a method described herein may be used to separate a plurality of ssDNA and a plurality of dsRNA. In some embodiments, a method described herein may be used to separate a plurality of a first dsDNA and a plurality of a second dsDNA. In some embodiments, a method described herein may be used to separate a plurality of dsDNA and a plurality of ssDNA. In some embodiments, a method described herein may be used to separate a plurality of dsRNA and a plurality of ssRNA.

In embodiments wherein the plurality of oligonucleotides includes dsDNA, the dsDNA may include linear dsDNA, circular dsDNA, looped dsDNA, or supercoiled dsDNA. In some embodiments, the plurality of oligonucleotides includes only one conformation of DNA (e.g., only linear dsDNA, only circular dsDNA, only looped dsDNA, only supercoiled dsDNA). In some embodiments, the plurality of oligonucleotides includes a mixture of two or more conformations of DNA.

In embodiments wherein the plurality of oligonucleotides includes ssRNA, the ssRNA may include linear ssRNA, circular ssRNA, looped ssRNA, or circular ssRNA. In some embodiments, the plurality of oligonucleotides includes only one conformation of RNA (e.g., only linear RNA, only circular ssRNA, only looped ssRNA, only supercoiled ssRNA). In some embodiments, the plurality of oligonucleotides includes a mixture of two or more conformations of RNA.

In some embodiments, the plurality of oligonucleotides comprises an oligonucleotide of from about 1 to 1000000 base pairs (e.g., from about 1000 to about 100000 base pairs). For example, nucleic acids to be separated by the present methods may be of from about 1000 base pairs to about 2000 base pairs, from about 2000 base pairs to about 3000 base pairs, from about 3000 base pairs to about 4000 base pairs, from about 4000 base pairs to about 5000 base pairs, from about 5000 base pairs to about 7500 base pairs, from about 7500 base pairs to about 10000 base pairs, from about 10000 base pairs to about 12500 base pairs, from about 12500 base pairs to about 15000 base pairs, from about 15000 base pairs to about 17500 base pairs, from about 17500 base pairs to about 20000 base pairs, from about 20000 base pairs to about 25000 base pairs, from about 25000 base pairs to about 30000 base pairs, from about 30000 base pairs to about 35000, form about 35000 to about 40000 base pairs, from about 40000 base pairs to about 45000 base pairs, from about 45000 base pairs to about 50000 base pairs, from about 50000 base pairs to about 55000 base pairs, from about 55000 base pairs to about 60000 base pairs, from about 60000 base pairs to about 65000 base pairs, from about 65000 base pairs to about 70000 base pairs, from about 70000 base pairs to about 75000 base pairs, from about 75000 base pairs to about 80000, from about 80000 base pairs to about 85000 base pairs, from about 85000 base pairs to about 90000 base pairs, from about 90000 base pairs to about 95000 base pairs, or from about 95000 base pairs to about 100000 base pairs.

### Mobile Phases and Injections

Methods described herein include eluting the sample from the liquid chromatography system by flowing a mobile phase through the liquid chromatography system. The components of a mobile phase (e.g., the solvent, the buffer, the ionic strength, etc.) are incredibly important factors to consider when performing a chromatography method. In some embodiments, a single mobile phase composition is used throughout the chromatography experiment. In such embodiments, the mobile phase is suitable for use in both affinity chromatography and slalom chromatography.

As the flow path between the affinity chromatography column and the slalom chromatography column is uninterrupted, the mobile phases used in the provided methods are compatible with both columns. Thus, the methods provide for direct affinity-slalom methods without solvent mismatch or the need for buffer exchange between modes of separation. Further, as the buffers used for both columns are the same, the disclosed methods eliminate or otherwise minimize the need for column equilibration between samples.

In some embodiments, a mobile phase may be characterized by one or more of a mobile phase buffer, a mobile phase pH, and a mobile phase flow rate. In some embodiments, the mobile phase buffer includes a weak signal in the detection method used. For example, in methods including UV detection, the mobile phase buffer may have a low UV absorbance signal. In some embodiments, the mobile phase buffer is tris(hydroxymethyl)aminomethane (“Tris”). In some embodiments, the mobile phase buffer is phosphate buffered saline. In some embodiments, the mobile phase buffer is present in the mobile phase at a concentration of from about 5 mM to about 500 mM. In some embodiments, the mobile phase pH is at least about 7 (e.g., at least 8, at least 9, at least 10, at least 11, at least 12, at least 13, at least 14). In some embodiments, the mobile phase pH is from about 7 to about 14 (e.g., from about 7 to 9 or from about 9 to about 12). In some embodiments, the mobile phase may be provided continuously (e.g., from a reservoir) at a controlled flow rate. In some embodiments, the mobile phase flow rate is from about 0.01 mL/min to about 5 mL/min (e.g., from about 0.1 mL/min to about 1 mL/min).

In some embodiments, one or more injections may be provided to the mobile phase (e.g., using an injector). The one or more injections may modify the composition of the mobile phase. For example, the method may include a sample injection and/or an elution injection.

The sample injection serves to introduce a sample (e.g., a sample containing a first group of oligonucleotides and a second group of oligonucleotides) to the affinity-slalom chromatography system. The sample injection includes the sample. In some embodiments, the sample injection is provided at a sample injection flow rate and a sample injection volume. In some embodiments, the sample injection flow rate is from about 0.01 mL/min to about 10 mL/min (e.g., from about 0.1 mL/min to about 1 mL/min, e.g., about 0.1 mL/min).

In some embodiments, the sample injection volume is from about 0.1 μL to about 100 μL (e.g., about 0.5 μL, about 1 μL, about 2 μL, about 3 μL, about 4 μL, about 5 μL, about 6 μL, about 7 μL, about 8 μL, about 9 μL, about 10 μL, about 20 μL, about 30 μL, about 40 μL, about 50 μL, about 60 μL, about 70 μL, about 80 μL, about 90 μL, about 100 μL, etc.). In some embodiments, the method includes providing at least one sample injection. In some embodiments, the method includes providing a series of successive sample injections. In some embodiments, the method includes providing from about 1 to about 1000 sample injections (e.g., from about 1 to about 100 injections, e.g., about 1 injection, about 10 injections, about 20 injections, about 30 injections, about 40 injections, about 50 injections, about 60 injections, about 70 injections, about 80 injections, about 90 injections, about 100 injections, etc.). In some embodiments, each sample injection includes a sample injection volume from about 0.1 μL to about 100 μL (e.g., about 0.5 μL, about 1 μL, about 2 μL, about 3 μL, about 4 μL, about 5 μL, about 6 μL, about 7 μL, about 8 μL, about 9 μL, about 10 μL, about 20 μL, about 30 μL, about 40 μL, about 50 μL, about 60 μL, about 70 μL, about 80 μL, about 90 μL, about 100 μL, etc.).

In some embodiments, a wait time may be provided after the sample injection, during which the mobile phase continues to flow at a wait time flow rate. The wait time may be included to allow the ensure the complete elution of the second group of oligonucleotides from the slalom chromatography column before elution of the first group of oligonucleotides from the affinity chromatography column directly onto the slalom chromatography column (also referred to as the “injection breakthrough” herein). In some embodiments, the wait time flow rate may be from about 0.01 mL/min to about 10 mL/min (e.g., from about 0.1 mL/min to about 1 mL/min, e.g., about 0.8 mL/min). In some embodiments, the wait time is from about 0.1 min to 20 min (e.g., 0.2 min, 0.3 min, 0.4 min, 0.5 min, 0.6 min, 0.7 min, 0.8 min, 0.9 min, 1 min, 1.5 min, 2 min, 2.5 min, 3 min, 3.5 min, 4 min, 4.5 min, 5 min, 6 min, 7 min, 8 min, 9 min, 10 min, 12 min, 14 min, 16 min, 18 min, 20 min, etc.).

The elution injection may include one or more components which reduce the affinity of one or more oligonucleotides retained by the affinity chromatography column. In some embodiments, these one or more additional components are selected to modify the pH of the mobile phase. In some embodiments, the one or more additional components include a base. In some embodiments, the base is ammonium bicarbonate or ammonium hydroxide. In some embodiments, the pH of the one or more injections is at least about 7 (e.g., at least 8, at least 9, at least 10, at least 11, at least 12, at least 13, at least 14). In some embodiments, the mobile phase pH is from about 7 to about 14 (e.g., from about 10 to about 11).

The elution injection is provided at a first elution injection flow rate and an elution injection volume. In some embodiments, the first elution injection flow rate is from about 0.01 mL/min to about 10 mL/min (e.g., from about 0.1 mL/min to about 1 mL/min). In some embodiments, the flow rate of the mobile phase is modified to a second elution injection flow rate after a second duration. The second elution injection flow rate may be from about 0.01 mL/min to about 10 mL/min (e.g., from about 0.1 mL/min to about 1 mL/min, e.g., about 0.8 mL/min). In some embodiments, the second duration is from 0 min to 1 min (e.g., about 0.1 min).

In some embodiments, the elution injection volume is from about 0.1 μL to about 100 μL (e.g., about 0.5 μL, about 1 μL, about 2 μL, about 3 μL, about 4 μL, about 5 μL, about 6 μL, about 7 μL, about 8 μL, about 9 μL, about 10 μL, about 20 μL, about 30 μL, about 40 μL, about 50 μL, about 60 μL, about 70 μL, about 80 μL, about 90 μL, about 100 μL, etc.). In some embodiments, the method includes providing at least one elution injection. In some embodiments, the method includes providing a series of successive elution injections. In some embodiments, the method includes providing from about 1 to about 1000 elution injections (e.g., from about 1 to about 100 injections, e.g., about 1 injection, about 10 injections, about 20 injections, about 30 injections, about 40 injections, about 50 injections, about 60 injections, about 70 injections, about 80 injections, about 90 injections, about 100 injections, etc.). In some embodiments, each elution injection includes an elution injection volume of from about 0.1 μL to about 100 μL (e.g., about 0.5 μL, about 1 μL, about 2 μL, about 3 μL, about 4 μL, about 5 μL, about 6 μL, about 7 μL, about 8 μL, about 9 μL, about 10 μL, about 20 μL, about 30 μL, about 40 μL, about 50 μL, about 60 μL, about 70 μL, about 80 μL, about 90 μL, about 100 μL, etc.).

In some embodiments, the elution time and peak resolution of one or more peaks may be modified by adjusting, e.g., the ionic strength of the mobile phase. In some embodiments, the affinity of one or more oligonucleotides to the affinity chromatography column is modified by adjusting, e.g., the ionic strength of the mobile phase.

### Zero and Low Volume Unions

The present technology and methods utilize an affinity chromatography column and a slalom chromatography column in direct fluidic connection. The direct fluidic connection can be achieved via a zero dead volume or low volume union. The zero and/or low volume union eliminates the need for the use of a valve switch or sample loop that are typically required to achieve separation of a sample by more than one chromatographic mode of separation.

A zero dead volume union introduces no volume (i.e., about 1 μL or less of volume) to the flow path. A low volume union introduces minimal volume (i.e., between about 0 to about 30 μL of volume) to the flow path.

Zero dead volume unions (also referred to as connectors) and low volume unions (also referred to as connectors) are known in the art. Examples of said unions include the VanGuard™ FIT (available from Waters Technologies Corporation, Milford MA) and as described in U.S. Pat. Nos. 8,449,769 and 9,724,621, incorporated herein by reference.

Additional examples include, but are not limited to, the Sulfinert Zero Dead Volume Union (available from Shimadzu), the Idex High-Pressure Zero Dead Volume Union (available from Fisher Scientific), and the Swagelok® Zero Dead Volume Union (available from Millipore Sigma).

Zero dead volume and low volume unions include male-male unions, female-female unions, and male-female unions, and can be used to connect the affinity chromatography column directly to the slalom chromatography column.

### Chromatography Systems

The chromatography systems described herein include an affinity chromatography column in direct fluidic connection to a slalom chromatography column. The direct fluidic connection between the affinity chromatography column and the slalom chromatography column is via a zero dead volume or low volume union. The chromatography system may further include a solvent pump, solvent mixer, a sample injector, and one or more detectors.

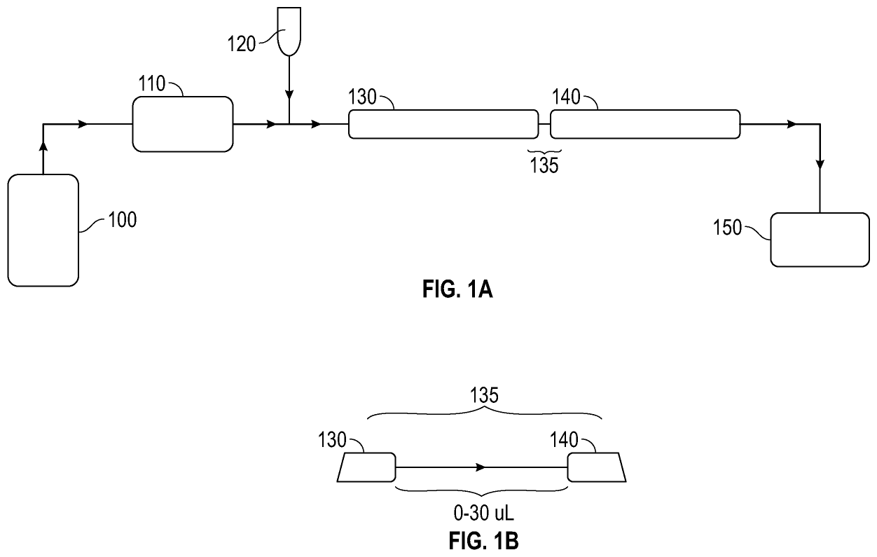

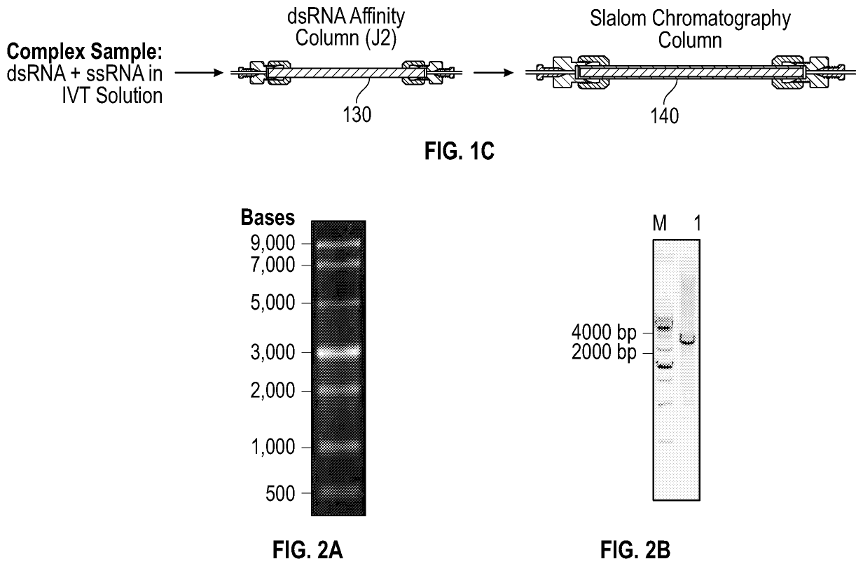

FIGS. 1A-1C depict an exemplary liquid chromatography (LC) system of the present technology. FIG. 1A demonstrates the LC system, with the flow path represented by the arrows. Solvent pump (100) pumps the solvent to the solvent mixer (110). A sample, which includes the target oligonucleotide, is loaded into the system using a sample injector (120) and flowed through the affinity chromatography column (130). The target oligonucleotide is eluted from the affinity chromatography column (130) directly onto the slalom chromatography column (140). Affinity chromatography column (130) and slalom chromatography column (140) are in direct fluidic connection via a zero dead volume or low volume union (135). Once eluted from the slalom chromatography column, the sample can be analyzed via a detector (150). For example, while FIG. 1A depicts a single detector (150), it is understood that the single detector (150) may be absent, or there may be multiple detectors connected in fluidic series.

FIG. 1B provides an enlarged view of the direct fluidic connection between the affinity chromatography column (130) and the slalom chromatography column (140). The flow path of 135 is between 0-30 μL of volume. In some embodiments, the flow path of 135 is less than 1 μL.

FIG. 1C depicts an exemplary union between the affinity chromatography column (130) and the slalom column (140).

The chromatography systems described herein allow for direct affinity-slalom chromatography in the absence of valve switching components. Accordingly, the present disclosure features chromatography systems without a valve switch along the flow path of the chromatography system. The chromatography systems described herein further allow for direct affinity-slalom chromatography in the absence of trap configurations. Accordingly, in some embodiments, a trap is absent from the flow path of the chromatography system.

In some embodiments, the slalom chromatography column (140) is connected in fluidic series to one or more detectors. Detectors suitable for use in the methods disclosed herein include detectors for ultraviolet spectroscopy, fluorescence spectroscopy, mass spectrometry, and/or other detectors used for the characterization of oligonucleotides, such as, for example, multi-angle light scattering (MALS) detectors and charged aerosol detectors (CAD). In some embodiments, the liquid chromatography system is connected in series to a detector for ultraviolet spectroscopy. In some embodiments, the liquid chromatography system is connected in series to a detector for fluorescence spectroscopy. In some embodiments, the liquid chromatography system is connected in series to detector for mass spectrometry. In some embodiments, the liquid chromatography system is connected in series to a MALS detector. In some embodiments, the liquid chromatography system is connected in series to a CAD. In some embodiments, the liquid chromatography system is connected to one or more of the detectors in series.

### Methods of Use

The methods disclosed herein pertain to direct affinity-slalom chromatography for the purification and characterization of an oligonucleotide from a sample. Methods of the present invention are capable of separating one or more oligonucleotides within a sample from one another during a chromatography experiment. Methods of the present invention feature the use of an affinity chromatography column to improve the separation of one class of oligonucleotides (e.g., ssRNA) from a second class of oligonucleotides (e.g., dsRNA), then directly eluting the eluent to a slalom chromatography column. The slalom chromatography column then separates a plurality of oligonucleotides within the first class of oligonucleotides into one or more distinct peaks (e.g., separating one or more different lengths of ssRNA molecules into one or more distinct peaks, each peak); each peak corresponding to a unique length of oligonucleotide. The class of oligonucleotide retained by the affinity chromatography column is then released from the affinity chromatography column and eluted onto the slalom chromatography column. The slalom chromatography column then separates a plurality of oligonucleotides within the second class of oligonucleotides into one or more distinct peaks (e.g., separating one or more different lengths of dsRNA molecules into one or more distinct peaks, each peak); each peak corresponding to a unique length of oligonucleotide.

Methods of the present disclosure include: (1) injecting a sample including one or more oligonucleotides onto an affinity chromatography column; (2) washing the affinity chromatography column with a mobile phase, thereby separating the one or more oligonucleotides of the sample into a first group of oligonucleotides that are retained by the affinity chromatography column, and a second group of oligonucleotides in the injection breakthrough that are eluted directly onto a slalom chromatography column; (3) eluting the injection breakthrough from the slalom chromatography column, thereby separating the one or more oligonucleotides of the injection breakthrough based on the principles of slalom chromatography; (4) releasing the one or more retained oligonucleotides from the affinity chromatography column directly onto the slalom chromatography column as an eluent; and (5) eluting the eluent from the slalom chromatography column, thereby separating the one or more retained oligonucleotides based on the principles of slalom chromatography.

In embodiments wherein the slalom chromatography column is connected in fluidic series to one or more detectors, the methods further include detecting the target oligonucleotide using the one or more detectors (e.g., a UV detector, a fluorescence detector, a MALS detector, and/or an MS detector). As such, the direct affinity-slalom chromatography methods described herein afford the analysis of a sample across two modes of separation, including the analysis of eluted oligonucleotides that do not bind to the affinity column; and peaks that elute after the release of the retained oligonucleotides from the affinity chromatography column (i.e., “elution peaks”).

An exemplary method of the present disclosure is now described. A sample including a plurality of oligonucleotides (e.g., a plurality of dsRNA and a plurality of ssRNA) is injected onto the affinity-slalom chromatography system at the affinity chromatography column. In some embodiments, the sample injection includes a sample injection volume from about 0.1 μL to about 100 μL (e.g., about 0.5 μL, about 1 μL, about 2 μL, about 3 μL, about 4 μL, about 5 μL, about 6 μL, about 7 μL, about 8 μL, about 9 μL, about 10 μL, about 20 μL, about 30 μL, about 40 μL, about 50 μL, about 60 μL, about 70 μL, about 80 μL, about 90 μL, about 100 μL, etc.). The sample injection further includes a sample injection flow rate, e.g., a sample injection flow rate of from about 0.01 mL/min to about 10 mL/min (e.g., about 0.1 mL/min to about 1 mL/min, e.g., about 0.8 mL/min). The oligonucleotides of the sample then separate into a first group of oligonucleotides and a second group of oligonucleotides based on their affinity to the affinity chromatography column. The first group of oligonucleotides have an affinity for the affinity chromatography column, and are therefore retained by the affinity chromatography column, whereas the second group of oligonucleotides elute from the affinity chromatography column directly onto the slalom chromatography column in the injection breakthrough. In some embodiments the second group of oligonucleotides do not have affinity for the affinity chromatography column. In some embodiments the second group of oligonucleotides have a weaker affinity for the affinity chromatography column than the first group of oligonucleotides. In some embodiments, the injection breakthrough may further include one or more additional components which do not have affinity for the affinity agent. For example, when the sample is prepared in a transcription medium, the injection breakthrough may include residual DNA with t7 promoter, T7RNA polymerase, one or more DNAse, pyrophosphates, nuclease, RNAse inhibitors, and one or more ribonucleotide bases.

The second group of oligonucleotides are then further separated (e.g., from each other and/or from one or more additional components in the injection breakthrough) by the high-flow conditions of the slalom chromatography column. The slalom chromatography column may separate the oligonucleotides in the second group into one or more peaks (e.g., a first injection breakthrough peak a second injection breakthrough peak, etc.), which in turn are detected by a detector down stream of the slalom chromatography column. In some embodiment, each peak includes a unique length of oligonucleotide within the second group of oligonucleotides.

In some embodiments, a wait time is provided after the sample injection, during which the mobile phase continues to flow through the column at a wait time flow rate. In some embodiments, the wait time flow rate is from about 0.01 mL/min to about 10 mL/min (e.g., about 0.1 mL/min to about 1 mL/min, e.g., about 0.8 mL/min). In some embodiments, the wait time is from about 0.1 min to about 20 min (e.g., about 0.2 min, about 0.3 min, about 0.4 min, about 0.5 min, about 0.6 min, about 0.7 min, about 0.8 min, about 0.9 min, about 1 min, about 1.5 min, about 2 min, about 2.5 min, about 3 min, about 3.5 min, about 4 min, about 4.5 min, about 5 min, about 6 min, about 7 min, about 8 min, about 9 min, about 10 min, about 12 min, about 14 min, about 16 min, about 18 min, about 20 min, etc.). The wait time may be selected as a time sufficient for all of the second group of oligonucleotides to elute from the affinity chromatography column to the slalom chromatography column, and from the slalom chromatography column to the detector. In some embodiments, the slalom chromatography column may be substantially devoid of the second group of oligonucleotides at the end of the wait time.

An elution injection is then provided to the affinity-slalom chromatography system at the affinity chromatography column. The elution injection includes a component (e.g., a base) which reduces the affinity of the first group of oligonucleotides to the affinity chromatography column. The first group of oligonucleotides therefore are released from the affinity chromatography column and directly elute onto the slalom chromatography column. In some embodiments, the elution injection includes an elution injection volume of from about from about 0.1 μL to about 100 μL (e.g., about 0.5 μL, about 1 μL, about 2 μL, about 3 μL, about 4 μL, about 5 μL, about 6 μL, about 7 μL, about 8 μL, about 9 μL, about 10 μL, about 20 μL, about 30 μL, about 40 μL, about 50 μL, about 60 μL, about 70 μL, about 80 μL, about 90 μL, about 100 μL, etc.). In some embodiments, the elution injection includes a first elution injection flow rate (e.g., an elution flow rate of from about 0.01 mL/min to about 10 mL/min (e.g., about 0.1 mL/min to about 1 mL/min, e.g., about 0.8 mL/min).

The first group of oligonucleotides are then further separated by the high-flow conditions of the slalom chromatography column. The slalom chromatography column may separate the oligonucleotides in the first group of oligonucleotides into one or more peaks (e.g., a first elution peak a second elution peak, etc.), which in turn are detected by the detector down-stream of the slalom chromatography column. In some embodiment, each peak includes a unique length of oligonucleotide within the first group of oligonucleotides.

It will be understood by those of skill in the art that this exemplary method is applicable to samples including one or more groups of oligonucleotides (e.g., a first group of oligonucleotides, a second group of oligonucleotides, a third group of oligonucleotides, a fourth group of oligonucleotides, etc.) and that each group of oligonucleotides may include one or more sizes of oligonucleotides (e.g., a first oligonucleotide size, a second oligonucleotide size, a third oligonucleotide size, a fourth oligonucleotide size, etc.).

### EXAMPLES

### Example 1: Separation of ssRNA and dsRNA Using Slalom Chromatography

The present example is directed to a comparative experiment, demonstrating the separation of a sample including a mixture of oligonucleotides using slalom chromatography alone, and direct affinity-slalom chromatography. In this exemplary experiment, the sample was formed by combining an ssRNA ladder with a dsRNA sample. The ssRNA ladder included RNA molecules of 500 bases, 1000 bases, 2000 bases, 3000 bases, 5000 bases, 7000 bases, and 9000 bases. A gel electrophoresis experiment highlighting the composition of the ssRNA ladder is shown in FIG. 2A. The dsRNA sample included a 4000 base pair RNA molecule, a 2000 base pair RNA molecule and a handful of unidentified components A gel electrophoresis experiment highlighting the composition of the dsRNA sample is shown in FIG. 2B. The ssRNA ladder and the dsRNA sample were mixed in a 50:50 ratio, a 10:90 ratio, a 5:95 ratio, and a 1:99 ratio.

A slalom chromatography experiment was performed to separate the oligonucleotides in the sample. The slalom chromatography experiment used a mobile phase include 10 mM Tris in water at pH 9. The sample was injected at a first injection flow rate of 0.1 mL/min from 0 min to 0.1 min; using an injection volume of 2 μL, and a total oligonucleotide concentration of 1 μg/μL. At 0.8 min, the flow rate was increased to a second injection flow rate 0.8 mL/min. The second flow rate was maintained until 5 minutes. The resulting chromatogram is shown in FIG. 3. The chromatogram clearly is made up of a plurality of ssRNA and dsRNA signals, with the ssRNA signals overlapping the dsRNA signals. In particular, the leading peak eluting at approximately 2.1 minutes decreases with decreasing dsRNA content, suggesting that at least this leading peak is the result of the 4000 base pair dsRNA molecule.

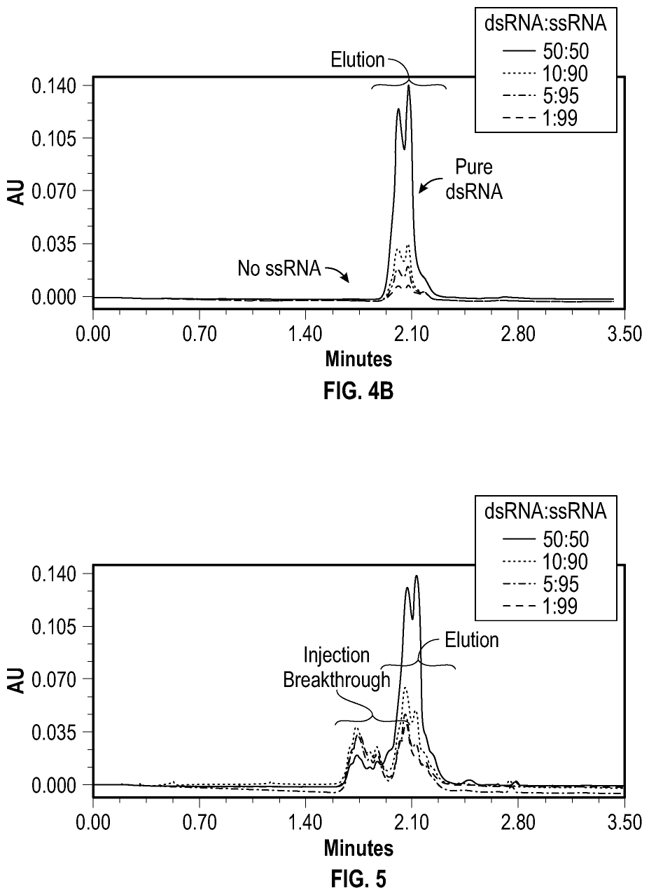

The experiment was then repeated, replacing the slalom chromatography column with a direct affinity-slalom chromatography column. The affinity chromatography column included an anti-dsRNA recombinant monobody affinity chromatography ligand. This method also utilized a mobile phase including 10 mM Tris in water at pH 9. The sample was injected at a first injection flow rate of 0.1 mL/min from 0 min to 0.1 min; using an injection volume of 2 μL, and a total oligonucleotide concentration of 1 μg/μL. At 0.8 min, the flow rate was increased to a second injection flow rate of 0.8 mL/min. The second injection flow rate was maintained until 2.61 minutes. The injection breakthrough was passed to a detector, resulting in the chromatogram shown in FIG. 4A. Then, an elution injection including 20 mM of ammonium bicarbonate was added to the chromatography system. The pH of the elution injection was adjusted to 10.6 by adding ammonium hydroxide. The elution injection was supplied at a first elution injection flow rate of 0.1 mL/min from 0 min to 0.1 min using an injection volume of 50 μL. At 0.11 min, the flow rate was increased to a second elution flow rate of 0.8 mL/min. The second elution flow rate was maintained until 3.5 min. The eluent from the elution injection was passed to a detector, resulting in the chromatogram shown in FIG. 4B.

As shown in FIG. 4A-4B, the direct affinity-chromatography experiment was able to separate ssRNA from dsRNA with high efficiency. All chromatogram peaks in the injection breakthrough map to ssRNA molecules from the ssRNA ladder. Similarly, in the eluent from elution injection, all chromatogram peaks map to dsRNA molecules from the dsRNA sample.

These results indicate that direct affinity-slalom chromatography methods show a distinct improvement over slalom chromatography alone in separating a first group of oligonucleotides (e.g., ssRNA) from a second group of oligonucleotides (e.g., dsRNA).

### Example 2: The Impact of Wait Time on the Performance of a Direct Affinity-Slalom Chromatography Experiment

The present experiment is directed to understanding the impact of wait time on the performance of a direct affinity-slalom chromatography experiment. The sample of Example 1, including a mixture of ssRNA and dsRNA in a 50:50 ratio, a 10:90 ratio, a 5:95 ratio, or a 1:99 ratio, was injected into the direct affinity-slalom chromatography column of Example 1 using a mobile phase including 10 mM Tris in water at pH 9. The sample was injected at a first injection flow rate of 0.1 mL/min from 0 min to 0.1 min; using an injection volume of 2 μL, and a total oligonucleotide concentration of 1 μg/μL. Then, an elution injection including 20 mM of ammonium bicarbonate added to the chromatography system. The pH of the elution injection was adjusted to 10.6 by adding ammonium hydroxide. The elution injection was supplied at a first elution injection flow rate of 0.1 mL/min from 0 min to 0.45 min using an injection volume of 50 μL. At 0.45 min, the flow rate was increased to a second elution flow rate of 0.8 mL/min. The second elution flow rate was maintained until 3.5 min. The eluent from the elution injection was passed to a detector, resulting in the chromatogram shown in FIG. 5.

This experiment lacked a second injection flow rate, and therefore provided no wait time between the sample injection and the elution injection. This in turn resulted in the injection breakthrough overlapping the elution, and the peaks from the ssRNA and the peaks from the dsRNA overlap in the chromatogram. Although there is overlap between ssRNA peaks and dsRNA peaks, it is notable that the overlap between the ssRNA peaks and the dsRNA peaks is reduced compared to the slalom chromatography only experiments.

These results indicate that although an immediate switch from a sample injection to an elution injection does aid in separating a first group of oligonucleotides (e.g., ssRNA) from a second group of oligonucleotides (e.g., dsRNA), the separation is improved by a wait time.

In general, a minimum wait time can ensure that late-eluting species (e.g., ~0.7 t₀ to 2 t₀) in the mRNA breakthrough do not overlap with the released and retained dsRNAs during elution. The timing is influenced by the impurity population present in the sample mixture and their respective behaviors on the slalom chromatography from 0.7 t₀ to 2 t₀).

1. A method of purifying a sample, the method comprising:
a) loading the sample onto an affinity chromatography column in direct fluidic connection to a slalom chromatography column, wherein the sample comprises a first group of oligonucleotides and a second group of oligonucleotides;
wherein the affinity chromatography column comprises:
a plurality of affinity chromatography particles, wherein the plurality of affinity chromatography particle comprises a plurality of nonporous polymer particles, wherein each particle within the plurality of nonporous polymer particles comprises a polymer core and a hydrophilic surface on an outer layer of the polymer core; and
one or more affinity agents conjugated to a surface of each particle of the plurality of affinity chromatography particles, wherein the first group of oligonucleotides comprises a stronger affinity for the one or more affinity agents than the second group of oligonucleotides;
and the slalom chromatography column comprises a plurality of slalom chromatography particles;
b) eluting the second group of oligonucleotides from the affinity chromatography column directly to the slalom chromatography column and retaining the first group of oligonucleotides in the affinity chromatography column in the presence of a mobile phase;
c) providing an elution injection to the affinity chromatography column, thereby releasing the first group of oligonucleotides from the affinity chromatography column;
d) eluting the first group of oligonucleotides from the affinity chromatography column directly to the slalom chromatography column in the presence of the mobile phase; and
e) detecting an injection breakthrough and an eluent from the slalom chromatography column at a detector;
wherein the injection breakthrough forms at least one distinct peak and the eluent form at least one distinct peak.

2. The method of claim 1, wherein the one or more affinity agents are conjugated directly to a surface of each particle of the plurality of affinity chromatography particles.

3. The method of claim 2, wherein the one or more affinity agents are conjugated to the surface of each particle of the plurality of affinity chromatography particles covalently.

4. The method of claim 1, wherein the one or more affinity agents are conjugated indirectly to the surface of each particle of the plurality of affinity chromatography particles through one or more conjugating molecules.

5. The method of claim 4, wherein the one or more conjugating molecules comprise streptavidin.

6. The method of claim 5, wherein the one or more affinity agents are biotinylated.

7. (canceled)

8. (canceled)

9. (canceled)

10. (canceled)

11. (canceled)

12. (canceled)

13. (canceled)

14. (canceled)

15. (canceled)

16. The method of claim 1, wherein the sample injection comprises a sample injection volume of from about 0.1 μL to about 100 μL.

17. (canceled)

18. The method of claim 1, wherein step b) comprises a first mobile phase flow rate of from about 0.1 mL/min to about 1 mL/min.

19. (canceled)

20. The method of claim 1, further comprising a wait time of from about 0.1 min to 20 min between steps b) and c).

21. (canceled)

22. (canceled)

23. (canceled)

24. The method of claim 1, wherein the injection breakthrough is detected prior to step c).

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The method of claim 1, wherein the eluent from the slalom chromatography column results in a zone retention factor of less than 0.35.

38. A chromatographic system comprising:
(a) an affinity chromatography column and a slalom chromatography column, wherein the affinity chromatography column and the slalom column are connected via a zero dead volume union, wherein the slalom column is positioned downstream of the affinity chromatography column; wherein the affinity chromatography column comprises:
a plurality of affinity chromatography particles, wherein the plurality of affinity chromatography particle comprises a plurality of nonporous polymer particles, wherein each particle within the plurality of nonporous polymer particles comprises a polymer core and a hydrophilic surface on an outer layer of the polymer core; and
and the slalom chromatography column comprises:
a plurality of slalom chromatography particles;
(b) a column injector positioned upstream of the affinity chromatography column; and
(c) tubing in fluidic connection with the slalom column positioned downstream of the slalom column.

39. The chromatographic system of claim 38, wherein the one or more affinity agents are conjugated directly to a surface of each affinity chromatography particle.

40. The chromatographic system of claim 39, wherein the one or more affinity agents are conjugated to the surface of each affinity chromatography particle covalently.

41. The chromatographic system of claim 38, wherein the one or more affinity agents are conjugated indirectly to the surface of each affinity chromatography particle through one or more conjugating molecules.

42. The chromatographic system of claim 41, wherein the one or more conjugating molecules comprise streptavidin.

43. The chromatographic system of claim 42, wherein the one or more affinity agents are biotinylated.

44. The chromatographic system of claim 38, wherein the one or more affinity agents comprise an oligonucleotide or an oligonucleotide specific antibody.

45. (canceled)

46. The chromatographic system of claim 44, wherein the oligonucleotide specific antibody is a J2 monobody.

47. (canceled)

48. The chromatographic system of claim 38, further comprising a detector in fluid communication with the tubing downstream of the slalom chromatography column.

49. (canceled)
